(19) United States
(12) Patent Application Publication
(10) Pub. No.: US 2002/0132758 A1
(43) Pub. Date: Sep. 19, 2002

(54) Method for identifying compounds to treat medical pathologies associated with molecular crystallization

(76) Inventor: John W. Shell, Hillsborough, CA (US)

Correspondence Address: REED & ASSOCIATES, 800 MENLO AVENUE, SUITE 210, MENLO PARK, CA, 94025, US

(21) Appl. No.: 10/052,712

(22) Filed: Jan. 17, 2002

Related U.S. Application Data
(60) Provisional application No. 60/262,987, filed on Jan. 18, 2001.

(51) Int. Cl. G01N 33/53; A61K 38/17

(52) U.S. Cl. 514/2; 435/7.1

(57) ABSTRACT

Numerous diseases and disorders are caused or exacerbated by the formation of crystalline aggregates of a biomolecule that is normally in solution. Such diseases and disorders include cataracts, sickle cell anemia, atherosclerosis, kidney stones, gallstones, gout, and Alzheimer's disease. The present invention provides methods to identify compounds that can inhibit the adverse formation of crystalline aggregates, including fibrils, of a target biomolecule. These methods include the screening of large combinatorial libraries. The identified compounds are tested for their therapeutic utility in treating medical conditions caused or exacerbated by the adverse crystallization of biomolecules. Molecules that are slight modifications of the target biomolecule are found to be particularly effective in inhibiting the adverse crystallization, including fibril formation, of a target biomolecule.

### CROSS-REFERENCE TO RELATED APPLICATIONS

This application claims priority under 35 U.S.C. §1 19(e)(1) to provisional U.S. Patent Application Serial No. 60/262,987, filed Jan. 18, 2001.

### TECHNICAL FIELD

This invention relates generally to inhibition of molecular crystallization processes associated with one or more medical pathologies, and more particularly relates to a method for screening compounds to identify potentially useful inhibitors of such crystallization processes. The invention additionally relates to a method of determining which, if any, of the identified potentially useful inhibitors are potentially useful as therapeutic agents by virtue of inhibiting a particular crystallization process. The invention also relates to a method for treating ocular cataracts by inhibiting the crystallization of one or more lenticular proteins.

### BACKGROUND

Crystalline materials consist of atoms that are regularly ordered in a periodically repeating pattern. At thermodynamic equilibrium, a crystal forms in preference to other possible phases when the total energy of the crystal is less than that of the phase (generally a fluid) from which the crystal grows. Crystals preferentially form because the crystalline phase has lower total energy than that of less-ordered phases, such as amorphous or glassy materials. The Second Law of Thermodynamics, however, requires permissible chemical reactions (including phase changes such as crystallization) to increase the total entropy (disorder) of the universe. Since a crystal is structurally more ordered than the fluid from which is grows, crystallization must proceed by creating a net entropy increase in the environment; this is accomplished by a release of heat (which increases disorder) into the surroundings. The formation of a crystal also requires that more heat be released (entropy increased) by crystallization than by the formation of a glassy or amorphous (non-crystalline) phase.

Crystallization is dependent not only upon thermodynamic principles but also upon reaction kinetics. A crystalline phase at thermodynamic equilibrium cannot form from a melt or solution unless several kinetic barriers are first crossed. The first barrier is that to nucleation: the formation of crystallographically ordered regions from a melt or solution (only solutions of molecules will be considered here). The nature of this barrier can be viewed by realizing that the total energy of a crystal is the sum of its surface energy and its internal energy. For large crystals, the surface energy is an insignificant part of the total. For the tiny crystals that first form in a solution, however, the surface energy can dominate, so that their total energy is greater than that of the solution. Excess energy (nucleation energy) is thus initially required for crystals to nucleate and grow, until a critical crystal radius is reached, at which point crystal growth starts releasing energy (heat) and proceeds spontaneously. Factors that affect nucleation include: the strength of the bonds formed between molecules, the concentration of the solution, the degree of cooling below the crystallization temperature (supercooling), the viscosity of the solution, and the size and shape of the crystallizing molecules. Very generally, stronger bonding will tend to stabilize nuclei and permit crystal growth; a high solute concentration will tend to favor nucleation; slight supercooling will favor nucleation, though greater supercooling may provide too little heat to overcome the nucleation energy barrier, resulting in the precipitation of amorphous material; a high solution viscosity may inhibit nucleation by reducing the diffusion and interaction of molecules, but may also serve to protect nuclei that have formed from dissolution; and large, complexly shaped molecules that must orient themselves in a highly specific manner with each other in order to crystallize will tend to have difficulty nucleating.

The other barriers to crystal growth considered here are the ability of molecules to be transported to the growing crystal, and the ability of heat to be transported away from the growing crystal. As a crystal grows, the immediately adjacent region of solution may become undersaturated, and growth may cease. The solution thus must permit rapid molecular diffusion and, eventually, it must be replenished with new molecules for crystal growth to continue. Similarly, if heat is not continuously removed from the growing crystal, crystal growth will cease.

It is thus appreciated that crystals will, in general, readily form from solutions of small molecules that bond strongly to each other; the bonding may be ionic, covalent, polar, or due to multiple hydrogen bonds. In the case of such molecules, crystallization will occur easily and spontaneously in a cooled supersaturated solution. For proteins and other large, complexly shaped molecules that tend to bond weakly to each other, crystallization will, in general, be more difficult because, among other factors, (1) the energy difference between the weakly bonded crystals and the solution from which they crystallize may be very small, and (2) the barriers to nucleation and crystal growth may be great.

Solvent molecules commonly become structurally incorporated during molecular crystallization. The resulting compounds (“solvates”), which are commonly less soluble than the corresponding solvent-free compounds, may crystallize or form other types of ordered structures, e.g., fibrils. When the ordered structure is crystalline and the solvent is water, the structure is termed a “crystalline hydrate.” The water molecules in hydrates are commonly incorporated stoichiometrically; thus monohydrates, dihydrates, hexahydrates, etc., are frequently formed, and these hydrates may be more thermodynamically stable (and less water-soluble) than the corresponding anhydrous forms of the compounds under certain conditions, e.g., physiologic conditions. That is, in the presence of water, and if free from certain barriers, the crystal hydrate will preferentially nucleate and grow rather than the anhydrous form. An example is cholesterol monohydrate, the crystalline material in atherosclerotic plaques.

Numerous medical pathologies are caused or exacerbated by the in vivo formation of crystalline material, including fibrils. Certain peptidyl compounds, including selected oligopeptides, polypeptides, and proteins, are known to form crystals and fibrils that are associated with various medical conditions, disorders, and diseases. Perhaps the best known example of such a disease is sickle cell anemia, which is caused by a single inherited point mutation in the gene coding for adult hemoglobin. The mutated protein (hemoglobin S) in the deoxygenated form is slightly less soluble than normal hemoglobin. As a result, it polymerizes and precipitates as fibers or crystals that distort and ultimately destroy the enclosing erythrocyte, with potentially severe medical consequences to the affected individual (see, e.g., Noguchi C. T. et al. (1985) “Sickle hemoglobin polymerization in solution and in cells, ” Annu Rev Biophys Biophys Chem 14:239-63; and Poyart, C. et al. (1996), “Hemolytic anemias due to hemoglobinopathies,” Mol Aspects Med 17:129-42).

In another example, amyloid peptides, particularly β-amyloid, are known to form ordered fibrillar aggregates that comprise the extracellular and cerebrovascular senile plaques associated with Alzheimer's disease. See Han et al. (1995), “The Core Alzheimer's Peptide NAC Forms Amyloid Fibrils which Seed and are Seeded by β-Amyloid: is NAC a Common Trigger or Target in Neurodegenerative Disease?” Chemistry and Biology 2:163-169; Serpell et al. (2000), “Molecular Structure of a Fibrillar Alzheimer's Aβ,” Biochemistry 39:13269-13275; Jarrett and Lansbury (1992), “Amyloid Fibril Formation Requires a Chemically Discriminating Nucleation Event: Studies of an Amyloidogenic Sequence from the Bacterial Protein OsmB,” Biochemistry 31(49):12345-12352; and Jarrett et al. (1993), “The Carboxy Terminus of the Beta Amyloid Protein is Critical for the Seeding of Amyloid Formation: Implications for the Pathogenesis of Alzheimer's Disease,” Biochemistry 32:4693-4697.

The prion diseases, e.g., the class of diseases known as the transmissible spongiform encephalopathies, are also characterized by abnormal protein deposition in brain tissue, in which the deposits are comprised of fibrillar amyloid plaques formed primarily from the prion protein (PrP). Such diseases include scrapie transmissible mink encephalopathy, chronic wasting disease of mule deer and elk, feline spongiform encephalopathy, and bovine spongiform encephalopathy (“mad cow disease”) in animals, and Kuru, Creutzfeldt-Jakob disease, Gerstmann-Struessler-Scheinker disease, and fatal familial insomnia in humans. It has been proposed that a 15-mer amino acid sequence, PrP96-111, is responsible for initiating prion formation in vivo by providing a seed for amyloid fiber formation. See Come et al. (1993), “A Kinetic Model for Amyloid Formation in the Prion Diseases: Importance of Seeding,” Proc Natl Acad Sc. USA 90:5959-5963. Fibrillin, associated with Martan's disease, is another example of a protein that forms an ordered fibrillar structure that causes an adverse medical condition. Fibrillar plaques formed from various collagens are also associated with certain medical pathologies, e.g., cardiac diseases and collagenofibrotic glomerulopathy; see Rossi et al. (2001), “Connective Tissue Skeleton in the Normal Left Ventricle and in Hypertensive Left Ventricle Hypertrophy and Chronic Chagasic Monocarditis,” Med Sci Mon 7:820-832; Yasuda et al. (1999), “Collagenofibrotic Glomerulopathy: A Systemic Disease,” Am J Kidney Dis 33:123-127.

Of course, peptidyl molecules can also form nonfibrillar crystalline structures, and these also commonly represent key components of plaques and deposits associated with medical pathologies. Such biomolecules include, without limitation: cystic fibrosis transmembrane conductance regulator (“CFTR”) protein, crystallization of which is associated with cystic fibrosis (see Berger et al. (2000), “Differences Between Cystic Fibrosis Transmembrane Conductance Regulator and HisP in the Interaction with the Adenine Ring of ATP,” J Biol Chem 275:29407-29412); phospholipases, which form Charcot-Leyden crystals associated with asthma, eosinophilic bone granuloma, eosinophilic pneumonia, and granulocytic leukemia (see Reginato and Kurnik (1989), “Calcium Oxalate and Other Crystals Associated with Kidney Diseases and Arthritis,” Semin Arthritis Rheum 18:198-224); cystine, which forms crystal deposits in bone marrow (associated with rickets and synovitis), the renal tubule and gastrointestinal tract (associated with cystinuria), and a variety of other body tissues, including the kidneys, eyes, and thyroid glands (associated with cystinosis, including the severe form of the disease, nephropathic cystinosis, or Fanconi's syndrome); and hemoglobin, hematoidin, cryoglobulins, and immunoglobulins (associated with hemarthrosis and other joint disorders, cryoglobulinemia, and multiple myeloma). See Gatter and Owen, Jr., “2. Crystal Identification and Joint Fluid Analysis,” in Gout, Hyperuricemia, and Other Crystal-Associated Arthropathies, Eds. Smyth et al. (New York: Marcel Dekker Inc., 1999), pp. 15-28; and Reginato and Kurnik, supra.

Lipids, particularly sterols and sterol esters, represent an additional class of biomolecules that form pathogenic crystalline deposits in vivo. Atherosclerotic plaque (atheroma) and cholesterol emboli are largely composed of cholesterol monohydrate and crystalline cholesteryl esters, including cholesteryl palmitate, oleate, linoleate, palmitoleate, linolenate, and myristate. See North et al. (1978), “The Dissolution of Cholesterol Monohydrate Crystals in Atherosclerotic Plaque Lipids,” Atherosclerosis 30:211-217; Burks and Engelman (1981), “Cholesteryl Myristate Conformation in Liquid Crystalline Mesophases Determined by Neutron Scattering,” Proc Natl Acad Sci USA 78:6863-6867; and Peng et al. (December 2000), “Quantification of Cholesteryl Esters in Human and Rabbit Atherosclerotic Plaques by Magic-Angle Spinning ¹³C-NMR,” Arterioscler Thromb Vasc Biol, pp. 2682-2688. Formation of gallstones is also associated with cholesterol crystallization, as gallstones commonly result from the crystallization of cholesterol monohydrate in bile. See Dowling (2000), “Review: Pathogenesis of Gallstones,” Aliment Pharmacol Ther 14 (Suppl. 2):39-46. Cholesterol crystals are associated with a host of additional medical pathologies, including rheumatoid arthritis, systemic lupus erythymatosis, anklosing spondylitis, bone cysts, bone granulomatosis (Erdheim-Chester disease), xanthomas, scleroderma, and paraproteinemia. Reginato and Falasca, “24. Calcium Oxalate and Other Miscellaneous Crystal Arthropathies,” in Gout, Hyperuricemia, and Other Crystal-Associated Arthropathies, supra. In the aforementioned reference, it was also proposed that crystalline deposits of other types of lipids, e.g., fatty acids, are pathogenic as well. See Reginato and Kurnik, supra. Cholesterol crystals are also observed in hypermature cataracts (e.g., Brooks, A. M. V. et al. (1994), “Crystalline nature of the iridescent particles in hypermature cataracts,” Br J Ophth 78:581-582; Knapp, H. C. (1937), “Spontaneous rupture of the lens capsule in hypermature cataract causing secondary glaucoma,” Am J Ophthalmol 20:820-821).

Researchers have determined that crystalline deposits of many other types of molecules, e.g., calcium salts and sodium salts, are responsible for numerous medical pathologies. Calcium oxalate, in the form of calcium oxalate monohydrate (“COM”; also referred to as “whewellite”), is the most common constituent of kidney stones. Lieske et al. (1994), “Renal Epithelial Cells Rapidly Bind and Internalize Calcium Oxalate Monohydrate Crystals,” Proc Natl Acad Sci USA 91:6987-6991. Kidney stones also contain crystals of other calcium compounds, including calcium oxalate dihydrate (“COD”; also referred to as “weddellite”) and “basic calcium phosphate” (either hydroxyapatite, octacalcium phosphate, or tricalcium phosphate.) See Ryall et al. (2000), “The Hole Truth: Intracrystalline Proteins and Calcium Oxalate Kidney Stones,” Mol Urol 4:391-402; Atmani et al. (1998), “Identification of Proteins Extracted from Calcium Oxalate and Calcium Phosphate Crystals Induced in the Urine of Healthy and Stone-Forming Subjects,” Urol Res 26:201-207; and Misra (2000), “Calcium and Disease: Molecular Determinants of Calcium Crystal Deposition Diseases,” Cell Mol Life Sci 57:421-428. Crystalline deposits of calcium salts are also associated with pseudogout (chondrocalcinosis), arthritis, and inflammation (see Gatter and Owen, supra, and Agudelo and Wise (1998), “Crystal-Associated Arthritis,” Clin Geriatr Med 14:495-513), and inflammation, wherein calcium pyrophosphate dihydrate (“CPPD”) and/or calcium hydrogen phosphate dihydrate (“CHPPD”) crystallize in the joints and/or tendons. CPPD crystallization has also been reported as a key factor in temporomandibular joint disease (“TMJ”); see Goudot et al. (1999), “A Destructive Calcium Pyrophosphate Dihydrate Deposition Disease of the Temporomandibular Joint,” J Craniofac Surg 10:385-388. Precipitated crystals formed of uric acid and uric acid salts (e.g., monosodium urate and monosodium urate monohydrate) are often found in the joints of arthritic patients and patients afflicted with gout. See McCarty (1994), “Crystals and Arthritis,” Dis Mon 40:225-299.

Although it has been confirmed that many medical pathologies are caused by, or exacerbated by, formation of molecularly ordered structures, this information has not been used to develop efficient methods for identifying compounds that can serve as inhibitors of crystallization or fibril formation, such compounds having potential utility as therapeutic agents. The present invention is directed, in part, to the aforementioned need in the art, and to providing a method for rapidly screening large numbers of compounds to identify those compounds that have potential utility in treating patients afflicted with or prone to a medical condition caused or exacerbated by adverse crystallization.

The present invention is also premised on the discovery that ocular cataract formation is accompanied by adverse crystallization of the lens proteins, and that human cataracts are themselves crystalline in nature. This very significant discovery now enables the identification of agents that are potentially useful in treating or preventing cataracts, as such agents will act as inhibitors of the crystallization process that leads to cataract formation.

Prior to the present invention, the only option for the restoration of sight in cataract patients has been the surgical removal of the opaque lens, a procedure that is usually accompanied by the implantation of a synthetic intraocular lens. The number of cataract operations in the United States alone approaches two million per year, the costs of which, particularly when surgery involves intraocular lens implantation, amount to a substantial financial burden on both patients and society. Moreover, cataract operations can be accompanied by adverse events such as corneal edema, secondary glaucoma, endophthalmitis due to infection, and delayed secondary capsule opacification. A straightforward therapeutic method for the prevention and treatment of all cataracts, as now provided by the present invention, represents an unprecedented advance in the field of ophthalmic medicine.

### SUMMARY OF THE INVENTION

It has now been discovered that the formation of many crystalline structures associated with adverse medical conditions can be inhibited by the presence of a molecular entity that (1) has sufficient structural similarity to the potentially crystallizing (including fibril-forming) molecule to have affinity therefor, but (2) inhibits crystal growth (or fiber formation) of the molecule. Without limiting the invention to a particular mechanism, it is frequently observed that molecules structurally similar to the potentially crystallizing molecules are found capable of isosteric replacement in the crystal nucleus that would otherwise form, but are incapable of supporting growth of that crystal lattice.

It is accordingly a primary object of the invention to provide a method for simultaneously screening a plurality of candidate compounds with respect to criterion (1), in order to identify those candidate compounds having affinity for a selected biomolecule that under normal circumstances forms a crystalline structure in vivo.

It is a related object of the invention to provide a method for determining which of the identified candidate compounds satisfy criterion (2) and therefore have potential utility as therapeutic agents.

It is another object of the invention to provide such methods wherein the target biomolecule is endogenous to the human body, and in vivo formation of the crystalline structure causes and/or exacerbates at least one medical pathology.

It is still another object of the invention to provide such methods wherein formation of the crystalline structure comprises fibril formation.

It is yet another object of the invention to provide such methods wherein the candidate compounds are selected so that they are structurally similar but nonidentical to the target biomolecule.

It is a further object of the invention to provide such methods wherein the plurality of candidate compounds is in the form of a combinatorial chemistry library in which each candidate compound is different.

It is still a further object of the invention to provide such methods wherein each candidate compound is prepared by chemically altering the target biomolecule, e.g., thermally, by oxidation, or by some other chemical reaction, to provide a potential crystallization inhibitor in the form of an analog of the target biomolecule.

It is still another object of the invention to provide such methods wherein each candidate compound is prepared by synthesizing structural analogs of an endogenous or other naturally occurring molecule that is known to be a crystallization inhibitor of the target biomolecule.

It is yet a further object of the invention to provide a method for treating a patient afflicted with a medical pathology associated with in vivo formation of a crystalline structure of a biomolecule that is endogenous to the human body, wherein the method involves administering to the patient a therapeutically effective amount of a crystallization inhibitor identified using the aforementioned methods.

It is an additional object of the invention to provide a method for preventing or treating cataract formation in the eye of a human patient by administering to the patient a therapeutically effective amount of an active agent effective to inhibit crystallization of at least one lenticular protein.

It is still an additional object of the invention to provide a method for identifying a compound capable of inhibiting formation of a pathogenic mass comprising a crystalline structure, by processing a surgically removed pathogenic mass to prepare a particulate suspension of the mass components and then screening the suspension against one or more candidate inhibitors.

### DETAILED DESCRIPTION OF THE INVENTION

I. Definitions and Nomenclature:

Before describing the present invention in detail, it is to be understood that this invention is not limited to specific active agents, dosage forms, dosing regimens, or the like, as such may vary. It is also to be understood that the terminology used herein is for the purpose of describing particular embodiments only, and is not intended to be limiting.

It must be noted that as used in this specification and the appended claims, the singular forms “a,” “an,” and “the” include plural referents unless the context clearly dictates otherwise. Thus, for example, reference to “a biomolecule” includes a single biomolecule as well as two or more biomolecules that may be the same or different, reference to “a candidate compound” includes combinations of two or more candidate compounds as well as a single candidate compound, and the like.

In describing and claiming the present invention, the following terminology will be used in accordance with the definitions set out below.

The term “biomolecule” refers to any organic or inorganic molecule, whether naturally occurring, recombinantly produced, or chemically synthesized in whole or in part, that is, was or can be a part of a living organism. The term encompasses, for example, nucleotides, amino acids, and monosaccharides as well as oligomeric and polymeric species such as oligonucleotides and polynucleotides; peptidic molecules such as oligopeptides, polypeptides and proteins; saccharides such as disaccharides, oligosaccharides, polysaccharides, mucopolysaccharides, peptidoglycans (peptido-polysaccharides), and the like. The term also encompasses lipidic molecules such as sterols (cholesterol, cholesterol monohydrate, cholesteryl esters, etc.), inorganic salts such as inorganic calcium salts (e.g., hydroxyapatite, octacalcium phosphate, tricalcium phosphate, calcium hydrogen phosphate dihydrate, calcium pyrophosphate, calcium pyrophosphate dihydrate), and organic acids and metal salts thereof (e.g., uric acid, monosodium urate, monosodium urate monohydrate, calcium oxalate, calcium oxalate monohydrate, etc.).

The terms “library” and “combinatorial library” are used interchangeably herein to refer to a plurality of chemical or biological moieties present on the surface of a substrate, wherein each moiety is different from each other moiety. The moieties may be, e.g., peptidic molecules and/or oligonucleotides.

The term “array” as used herein refers to a two-dimensional arrangement of features, such as an arrangement of different molecules on a substrate surface (e.g., as in an oligonucleotide or peptidic array), wherein the molecules are attached to the surface, either covalently or noncovalently, wherein the attachment may be direct, or, more commonly, indirect, i.e., through a linkage that provides a spacer between the substrate surface and the biomolecule. Noncovalent attachment will typically involve “adsorption” such as may occur through hydrogen bonding, van der Waal's forces, polar attraction, electrostatic forces (i.e., through ionic bonding), or the like. Arrays are generally comprised of similar features ordered in, for example, a rectilinear grid, but non-ordered arrays may be used as well. An array is distinguished from the more general term “pattern” in that patterns do not necessarily contain similar and ordered features.

The term “candidate compound” refers to a compound that may or may not have a desired property, which in the present case is the ability to physically associate with a particular biomolecule that forms a pathogenic ordered structure in vivo.

The terms “crystalline,” “crystalline structure,” “ordered structure,” or “molecularly ordered structure” as used herein refer to a plurality of molecules that are physically associated with each other in a regular manner that involves alignment along one or more axes. Such structures are normally formed from a plurality of identical molecules. These structures include fibrillar structures.

The terms “peptide, ” “peptidyl, ” and “peptidic” as used throughout the specification and claims are intended to include any structure comprised of two or more amino acids. The amino acids forming all or a part of a peptide may be any of the twenty conventional, naturally occurring amino acids, i.e., alanine (A), cysteine (C), aspartic acid (D), glutamic acid (E), phenylalanine (F), glycine (G), histidine (H), isoleucine (I), lysine (K), leucine (L), methionine (M), asparagine (N), proline (P), glutamine (Q), arginine (R), serine (S), threonine (T), valine (V), tryptophan (W), and tyrosine (Y). Any of the amino acids may be replaced by a non-conventional amino acid such as, for example, an isomer or analog of a conventional amino acid (e.g., a D-amino acid), non-protein amino acids post-translationally modified amino acids enzymatically modified amino acid, a construct or structure designed to mimic an amino acid (e.g., an α,α-disubstituted amino acid, N-alkyl amino acid, lactic acid, β-alanine, naphthylalanine, 3-pyridylalanine, 4-hydroxyproline, O-phosphoserine, N-acetylserine, N-formylmethionine, 3-methylhistidine, 5-hydroxylysine, and nor-leucine). Peptidic compounds herein also include compounds wherein the naturally occurring amide —CONH— linkage is replaced at one or more sites within the peptide backbone with a non-conventional linkage such as an N-substituted amide, ester, thioamide, retropeptide (—NHCO—), retrothioamide (—NHCS—), sulfonamido (—SO₂NH—), and/or peptoid (N-substituted glycine) linkage. Accordingly, peptidic molecules herein include pseudopeptides and peptidomimetics. The peptides of this invention can be (a) naturally occurring, (b) produced by chemical synthesis, (c) produced by recombinant DNA technology, (d) produced by biochemical or enzymatic fragmentation of larger molecules, (e) produced by methods resulting from a combination of methods (a) through (d) listed above, or (f) produced by any other means for producing peptides.

The term “therapeutic agent” is used herein to refer to any chemical compound, complex, or composition that is suitable for administration to a mammalian patient and that has a beneficial biological effect, which may be a prophylactic effect and/or a therapeutic effect, with respect to a disease or abnormal physiological condition associated with in vivo formation of an ordered structure.

The terms “treating” and “treatment” as used herein refer to methods to reduce the severity and/or frequency of symptoms, eliminate an underlying cause, prevent the occurrence of symptoms and/or their underlying cause, and improve or remediate damage. Thus, for example, “treating” a patient involves applying methods to prevent a particular disorder or adverse physiological event in a susceptible individual, as well as applying methods to inhibit or cause regression of a disorder or a disease in a clinically symptomatic individual.

By an “effective” amount of a therapeutic agent is meant a nontoxic but sufficient amount of the agent to provide the desired effect.

The term “pharmaceutically acceptable,” such as in the recitation of a “pharmaceutically acceptable carrier,” refers to a material that is not biologically or otherwise undesirable, i.e., the material may be incorporated into a pharmaceutical composition administered to a patient without causing any undesirable biological effects or interacting in a deleterious manner with any of the other components of the pharmaceutical composition. “Pharmacologically active” (or simply “active”), as in a “pharmacologically active ” derivative, refers to a material having the same type of pharmacological activity as the parent compound, to an approximately equivalent degree. When the term “pharmaceutically acceptable” is used to refer to a derivative (e.g., a salt) of an active agent, it is to be understood that the compound is pharmacologically active as well. When the term, “pharmaceutically acceptable” is used to refer to an excipient, it implies that the excipient has met the required standards of toxicological and manufacturing testing or that it is included in the Inactive Ingredient Guide prepared by the FDA.

“Optional” or “optionally” means that the subsequently described circumstance may or may not occur, so that the description includes instances where the circumstance occurs and instances where it does not.

The term “substantially” as in, for example, the phrase “substantially all molecules of an array,” refers to at least 90%, preferably at least 95%, more preferably at least 99%, and most preferably at least 99.9%, of the molecules of an array. Other uses of the term “substantially” involve an analogous definition.

II. Inhibition of Pathological Crystallization

Crystallization begins with the nucleation of an ordered domain of atoms or molecules that then can act as a template for the addition of further atoms or molecules. Nucleation can be homogeneous, occurring directly from solution, or heterogeneous, occurring on the surface of a material that is in contact with the solution. The great majority of nucleation events are heterogeneous; homogeneous nucleation in solution is, in fact, usually difficult to produce and may require a high degree of supercooling. Heterogeneous nucleation is favored on surfaces that have some structural similarity to the nucleating crystal. For example, the normal structures of crystalline biomineralized tissues, such as bones and teeth, are determined in large part by interactions between protein templates (e.g., collagens) and the crystals that grow on them (see, e.g., the review by Addadi, L. et al., (2001), “On how proteins interact with crystals and their effect on crystal formation”, Z Kardiol 90:81-92). The templates can influence the size, morphology, orientation, and even the atomic structure of the resulting crystals.

Conversely, endogenous proteins and other compounds are used by the body to prevent potentially harmful crystal nucleation and growth. For example, the crystallization of cholesterol from bile is inhibited in normal individuals by endogenous proteins (e.g., Secknus, R. et al., (1996), “Purification and characterization of a novel human 15 kd cholesterol crystallization inhibitor protein in bile”, J Lab Clin Med 127:169-178; and Ohya, T. et al. (1993), “Isolation of a human biliary glycoprotein inhibitor of cholesterol crystallization,” Gastroenterology 104:527-538). In pathological conditions in which such an inhibitory protein is lacking, cholesterol may crystallize to form gallstones. Similarly, endogenous proteins prevent the crystallization of calcium oxalate in the kidneys, thus inhibiting the formation of calcium oxalate kidney stones (e.g., Atmani, F. et al. (1996) “Inter-alpha-inhibitor: a protein family involved in the inhibition of calcium oxalate crystallization,” Scanning Microsc 10:425-433; and Selvam, R. et al. (2000) “A novel basic protein from human kidney which inhibits calcium oxalate crystal growth,” BJU Int 86:7-13.)

In general, successful crystal nucleation requires the presence of either a substrate that stabilizes the nuclei as they form, or conditions that permit homogeneous nucleation. The conditions for homogeneous nucleation of complex molecular crystals are only expected to be met rarely in vivo: in cases of a supersaturated, nearly pure solution that is slightly supercooled and possesses sufficient energy to overcome the nucleation energy barrier. Heterogeneous nucleation is expected to be far more common, due to the abundance of proteins and other large molecules that may provide structurally compatible templates for crystal nucleation.

Once a crystal nucleus exceeds the critical radius (as described above), further crystal growth depends upon a steady supply of new molecules of the same kind to the crystal and the continued dissipation of heat from the crystal. In the case of proteins and other large, complex molecules, crystallization must proceed slowly enough to allow molecules that approach the growing crystal to properly orient themselves as they become incorporated into the crystal; rapid solidification may result in a high degree of disorder and lack of crystallinity.

The present invention is based on the discovery that molecular crystal growth can be prevented by the presence of a structurally distinct molecule that nevertheless has affinity for the potentially crystallizing molecule. The crystallization-inhibiting molecule in some cases differs only slightly in structure from the crystallization-inhibited molecule; such molecular pairs have affinity for each other, but their structural differences prevent the formation of ordered molecular structures such as crystals or fibrils. Crystallization-inhibiting molecules prevent crystallization by interfering with crystal nucleation and/or post-nucleation crystal growth. The invention is specifically directed to the application of this discovery to the identification of molecules that inhibit the crystallization of biomolecules in cases where such crystallization causes or aggravates adverse medical conditions.

III. Preparation of Crystallization-Inhibiting Molecular Candidates

Any method for producing one or more molecules that can serve as potential crystallization-inhibiting candidate molecules is included in this invention. Preferred methods include: (1) selection of particular candidate compounds based upon computer modeling of structural interactions between candidate compounds and a target biomolecule for which crystallization prevention is desired, (2) the use of such modeling to design a plurality of candidate crystallization-inhibiting molecular compounds that are then synthesized, (3) variously modifying the target biomolecule to create a plurality of candidate crystallization-inhibiting molecules, and (4) variously modifying a molecule endogenous to the human body or that otherwise occurs naturally that is known to inhibit crystallization of a biomolecule to create a plurality of candidate crystallization-inhibiting molecules.

The ready availability of powerful high-speed computers and of databases containing tens of thousands of crystal structures (such as the Cambridge Structural Database (Cambridge Crystallographic Data Centre, Cambridge, United Kingdom) and the Protein Data Bank (Research Collaboratory for Structural Bioinformatics, Piscataway, N.J.)) greatly facilitates the modeling of potential structural interactions among molecules. Such modeling is routinely used to screen potential drug candidates for interactions with target biomolecules, such as specific regions of particular proteins or specific DNA sequences (see, e.g., the review by Mason, J.S. et al. (2001), “3-D pharmacophores in drug discovery”, Curr Pharm Des 7:567-97). These computer-modeling methods can be used in conjunction with the present invention to identify crystallization-inhibiting candidate molecules. Such candidate molecules are identified by their ability, as predicted by computer-modeling techniques, to bind or otherwise closely interact with a target biomolecule, but be structurally distinct from it. To accomplish this identification, regions of the molecules are sought that fit together such that interatomic distances are close enough to allow bonding (such as hydrogen bonding or van der Waal's bonding), but not so close that repulsive forces become dominant, and also such that atoms on the two molecules are aligned to allow hydrogen-bonding or other types of bonding.

Once the surface conformation and potential bonding sites on a target molecule are identified, computer programs also can be used to design plausible structures of candidate molecules that could bind with or otherwise closely interact with the target molecule. These molecules can then be synthesized and used as crystallization-inhibiting candidates.

Another preferred method to generate a plurality of candidate crystallization-inhibiting molecules is to modify the target biomolecule. Since molecules that are slight structural modifications of the target molecule are likely to physically interact with the target molecule, a fruitful strategy for generating crystallization-inhibiting molecular candidates is to create a number of molecules that structurally differ only slightly from the target molecule. These generated molecular analogs to the target biomolecule each have at least one difference in molecular structure relative to the unmodified biomolecule but are otherwise structurally identical to it. This strategy may be implemented by subjecting the target biomolecule to a series of conditions that slightly degrade the target molecule or otherwise alter it. Such conditions include, without limitation, exposing the target biomolecule to heat (preferably by subjecting the substance in the dry state to a temperature just below the melting point of the substance), a change in pH, ionizing radiation such as ultraviolet radiation or x-rays, intense visible light, oxidizing or reducing conditions, or other chemical reactants. As further described in Sections IV and V below, each generated analog is tested as an inhibitor of the pathogenic crystallization by contacting the target biomolecule with the analog under conditions effective to facilitate formation of the crystalline structure in the absence of any crystallization inhibitors, and then determining whether the crystalline structure is formed (e.g., by microscopic observations in polarized light or by the use of x-ray diffraction techniques). If the crystalline structure has not been formed, the analog is identified as a compound capable of inhibiting formation of said crystalline structure.

As an example, a compound with a close structural similarity to cholesterol can be generated by heating pure cholesterol in the dry state at a temperature slightly below the melting point of the pure substance. The presence of the resulting material in an aqueous solution of cholesterol has been found to significantly increase the measurable water solubility of pure cholesterol from its normal value. This observation is consistent with the conclusion that a structurally similar material can inhibit the nucleation and growth of crystals from a solution.

A further preferred method of generating potential crystallization-inhibiting molecules is to synthesize structural analogs of a molecule endogenous to the healthy human body, or that otherwise occurs in nature, that is known to inhibit the adverse crystallization of a biomolecule, where each analog is related to the natural molecule by at least one structural difference. One method of generating such analogs is to extract and purify the natural compound, and then subject it to conditions likely to create small molecular changes. Such conditions include those described above, e.g., exposing the natural compound to heat, a change in pH, ionizing radiation such as ultraviolet radiation or x-rays, intense visible light, oxidizing or reducing conditions, or other chemical reactants. Another method of generating analogs is by using well-known recombinant techniques. For example, the gene (or genes) coding for the natural compound is isolated and one or more small changes are made to the coding sequence. The gene is then inserted into a bacterium or other organism that is induced to express the product of the gene, which is then isolated and tested as a potential candidate compound. This process can be repeated to test many possible alterations to the gene, and thus many analogs of the natural molecule. Other means to synthesize analogs to the natural molecule will be apparent to those skilled in the art of synthetic chemistry any such methods can be used in conjunction with this invention. The synthesized molecules are then screened for their possible utility in inhibiting the crystallization of a target biomolecule, as described in the following sections.

IV. The Use of Combinatorial Libraries to Screen Compounds for Interaction with a Target Biomolecule

An alternate strategy for identifying compounds that may inhibit the crystallization of a target biomolecule is to create a large library of compounds and allow each of these compounds to interact with the target biomolecule. Interactions, particularly hybridizations, between the target biomolecule and the library of test compounds are observed and recorded. The libraries typically contain tens to millions of compounds, or more. The library compounds may be structurally diverse or, preferably, they may represent a multitude of structural permutations of a parent structure, preferably the target biomolecule. Such libraries wherein all the compounds are different, referred to as “combinatorial libraries” or “compound libraries,” may be comprised of commercially available compounds, compounds custom synthesized commercially or by the researcher, or any combination of these. The library compounds are typically densely arrayed on a substrate. Several commercial firms custom prepare such densely packed combinatorial library arrays; such firms include Tripos Inc. (St. Louis, Mo.); ChemNavigator Inc. (San Diego, Calif.); ArQule, Inc. (Woburn, Mass.); Pharmacopoeia, Inc. (Princeton, N.J.); Trega Biosciences, Inc. (San Diego, Calif.); and Ambinter (Paris, France).

Combinatorial libraries can also be prepared using now-conventional means for attaching the compounds to discrete sites on a substrate surface using, for example, photolithography techniques (e.g., U.S. Pat. No. 5,143,854 to Pirrung et al.) or electrochemical patterning of a substrate surface (e.g., U.S. Pat. No. 5,667,667 to Southern).

Thus, in a first preferred embodiment of the invention, numerous candidate compounds are simultaneously screened for their ability to inhibit crystallization of a selected biomolecule that is endogenous to the human body, wherein the in vivo crystallization is an adverse event resulting in at least one medical pathology selected from diseases, disorders, and other undesirable physiological conditions. The resulting crystalline structure may be fibrillar in nature. The method involves use of a combinatorial library of a plurality of different candidate compounds each attached to a different site on a substrate. The library will typically but not necessarily be in the form of an ordered array, and suitable libraries may be obtained commercially or be chemically synthesized using conventional surface attachment chemistry known to those skilled in the art of solid phase synthesis and/or array preparation. The combinatorial library of candidate compounds is contacted with the selected biomolecule under conditions that allow for and preferably facilitate its crystallization in the absence of any inhibitors. It will be appreciated that such conditions include, by way of example, concentration, pH, time, temperature, and ionic strength.

Candidate compounds for which the biomolecule has affinity are then identified by determining which candidate compounds within the combinatorial library have become physically associated with the biomolecule. This may be done by using a labeled biomolecule, wherein the selected biomolecule is labeled with a detectable label prior to use, e.g., a fluorescent label (which is preferred), an ultraviolet label (i.e., a label identified by using ultraviolet spectroscopy), a colored label, or the like. Affinity also may be detected by measuring the refractive indices of the solutions before and after the interactions, as the refractive index produced by a bound molecule in general will differ from that of an unbound molecule. In cases where the target biomolecule, generally a protein, is capable of unfolding due to a thermal change (e.g., heating), affinity may be measured using the method of Pantoliano et al. (U. S. Pat. No. 6,268,158). In this method, a physical change associated with the denaturation of the target biomolecule (e.g., turbidity of a solution of the target biomolecule measured at 350 mn) is measured as a function of temperature. Measurements are made for the pure biomolecule and for the combinations of the target biomolecule with the test compounds. In cases where the test compound has affinity for the target biomolecule, the denaturation curve is altered relative to that for the pure target biomolecule. For any of these methods, or for any other affinity-detecting method used, the candidate compounds (i.e., those that show affinity to the target biomolecule) are selected as “lead” compounds, i.e., as potential inhibitors of in vivo formation of a crystalline structure of the biomolecule.

Evaluations of the interactions between library compounds and a target biomolecule may be accomplished using high-throughput screening methods. Such methods are routinely employed by the pharmaceutical industry and are well known to those skilled in the art of pharmaceutical discovery and development (see, e.g., reviews by Hertzberg, R. P. et al. (2000), “High-throughput screening: new technology for the 21^{st} century,” Curr Opinion Biotech 4:445-451; and Olsen, M. et al. (2000), “High-throughput screening of enzyme libraries,” Curr Opinion Biotech 4:331-337). These methods, which employ robotics and microtiter techniques, allow as many as 100,000 assays to be performed per day, or even more (Hertzberg et al., supra). The preferred labeling method for these techniques is fluorescence. Fluorescence intensity is generally measured, which can indicate the presence or absence of hybridization, though fluorescence anisotropy (measuring polarization of the fluorescent signal) is sometimes used, which can provide information on mass.

V. The Use of Combinatorial Libraries to Identify Inhibitors of Molecular Crystallization

Once candidate compounds are identified by their ability to become physically associated with a target biomolecule, the present invention provides methods for testing whether the candidate compounds inhibit crystallization of the biomolecule under specified conditions (e.g., physiologic conditions). Thus, in a further embodiment of the invention, the lead compounds are evaluated so that one or more crystallization inhibitors can be identified. This evaluation is effected by carrying out the steps of the preceding section as described above, and the biomolecule is then contacted with each lead compound under conditions effective to allow for and preferably facilitate its crystallization in the absence of any inhibitors. The conditions, for example, may be those that approximate physiologic conditions, such as a temperature of approximately 37° C. and a pH of approximately 7.4. If a crystalline structure is not formed in the presence of a particular lead compound, then that compound is identified as one that is capable of inhibiting crystallization of the target biomolecule. High-throughput screening methods, such as those already referred to, may be employed for these assays and evaluations. Once a compound has been identified as a crystallization inhibitor (a lead compound), the compound can be further evaluated as a therapeutic agent by measuring key pharmacokinetic parameters such as absorption, metabolism, excretion, dosage, in vivo efficacy, and toxicity. These parameters may be measured, according to methods well known in the art of pharmaceutical development, in laboratory animals (e.g., mice, rats, dogs, monkeys) and in human subjects. In general, a compound will first be tested in an animal model of the pathology to be treated, followed by the compound being tested in human subjects who are affected by the pathology or are susceptible to being affected by the pathology.

In a related embodiment, lead compounds are identified and evaluated with respect to their ability to inhibit formation of a pathogenic mass comprising a crystallline structure, wherein the candidate compounds are not screened with respect to a single biomolecule but rather against the components of the pathogenic mass as a whole. This is done by using a particulate suspension of the components of the pathogenic mass, prepared by obtaining a pathogenic mass that has been freshly removed, surgically, from a human patient, then lyophilizing and triturating the mass, and finally admixing the lyophilized triturated mass with an inert organic solvent, e.g., dimethyl sulfoxide (“DMSO”) or the like. The pathogenic mass may be, for example, a cataract, an atherosclerotic plaque, a neuritic plaque, a dendritic plaque, a gallstone, a kidney stone, or the like. The combinatorial library of candidate compounds is contacted with the particulate suspension under conditions effective to facilitate crystallization in the absence of any crystallization inhibitors, and candidate compounds for which one or more molecular components of the pathogenic mass have affinity are identified by determining which candidate compounds have become physically associated with a component of the particulate suspension. The methods used to determine physical association are, e.g., those discussed in the preceding section. Any identified compounds are selected as lead compounds for further evaluation.

A preferred embodiment of the present invention involves pharmaceutical formulations that comprise a molecule effective to inhibit the pathological crystallization of a biomolecule. The formulations are prepared according to methods well known in the art of drug formulation, as described in the pertinent literature, textbooks, and reference books (e.g., Remington: The Science and Practice of Pharmacy, 20^{th} Edition, Gennaro, A. R., Ed. (Lippincott, Williams and Wilkins, 2000)).

The compounds of the invention may be administered orally, parenterally, rectally, buccally, sublingually, nasally, vaginally, transurethrally, by inhalation, topically, transdermally, topically to the eye, intraocularly, or via an implanted reservoir, in dosage forms containing conventional pharmaceutically acceptable carriers and excipients. The term “parenteral” as used herein is intended to include subcutaneous, intravenous, and intramuscular injection. Topical ocular and particularly oral dosage forms are preferred. The amount of the compound administered will, of course, be dependent on the particular active agent, the condition or disorder being treated, the severity of the condition or disorder, the subject's weight, the mode of administration, and other pertinent factors known to the prescribing physician. Generally, however, dosage will be in the range of approximately 0.001 mg/Kg/day to 100 mg/Kg/day, more preferably in the range of about 0.1 mg/Kg/day to 10 mg/Kg/day.

Suitable compositions and dosage forms include tablets, capsules, caplets, gel caps, troches, dispersions, suspensions, solutions, syrups, transdermal patches, gels, powders, magmas, lozenges, creams, pastes, plasters, lotions, discs, suppositories, liquid sprays for nasal or oral administration, dry powder, aerosolized formulations for inhalation, solutions and ointments for topical ocular administration, solutions for intraocular administration, and the like.

Depending on the intended mode of administration, the pharmaceutical formulation may be a solid, semi-solid, or liquid, such as, for example, a tablet, a capsule, caplets, a liquid, a suspension, an emulsion, a suppository, granules, pellets, beads, a powder, or the like, preferably in unit dosage form suitable for single administration of a precise dosage. Suitable pharmaceutical compositions and dosage forms may be prepared using conventional methods known to those in the field of pharmaceutical formulation and described in the pertinent texts and literature, e.g., in Remington: The Science and Practice of Pharmacy, 20^{th} Edition, Gennaro, A. R., Ed. (Lippincott, Williams and Wilkins, 2000).

VII. Treatable Medical Conditions

Any medical condition, disease, or disorder that is caused or aggravated by the crystallization, including fibril formation, of a biomolecule can be prevented or treated by the compositions and methods of this invention. The pathologies listed in the following table are only a few examples of medical conditions that can be so prevented or treated, and it should in no way be construed that the invention is limited to the treatment or prevention of these pathologies.

| | Biomolecule that Undergoes |
|---|---|
| | Pathological Crystallization or |
| Medical Condition | Fibril Formation |
| Cataract | Crystallins |
| | Cholesterol (rare) |
| | Connexins |
| Sickle cell anemia | Hemoglobin S |
| Alzheimer's disease | Amyloids, particularly β-amyloid |
| Transmissible spongiform | Amyloids, particularly prion protein |
| encephalopathies (e.g., | (PrP) |
| scrapie, Cruetzfelt-Jakob | |
| disease, and Gerstmann- | |
| Straussler-Scheinker disease) | |
| Martan's disease | Fibrillin |
| Collagenofibrotic glomerulo- | Fibrillin |
| pathy | |
| Cystic fibrosis | Cystic fibrosis transmembrane |
| | conductance regulator |
| Hemarthrosis, cryoglobulinemia, | Hemoglobin, hematoidin, cryoglobulins, |
| multiple myeloma | immunoglobulins |
| Asthma, eosinophilic bone | Phospholipases (Charcot-Leyden |
| granuloma, eosinophilic | crystals) |
| pneumonia, granulocytic | |
| leukemia | |
| Gout (hyperuricemia) | Uric acid, monosodium urate, |
| | monosodium urate monohydrate |
| Pseudogout (chondrocalcinosis) | Calcium pyrophosphate dihydrate |
| Kidney stone (nephrolithiasis; | Calcium oxalates |
| urolithiasis) | Whewellite |
| | Weddellite |
| | Calcium phosphates |
| | Hydroxyapatite |
| | Tricalcium phosphate |
| | Octacalcium phosphate |
| | Cystine |
| | Uric acid |
| | Struvite [(NH₄)MgPO₄ · 6(H₂O)] |
| Gallstone | Cholesterol monohydrate |
| | Bilirubin |
| Atherosclerosis | Cholesterol monohydrate |
| | Cholesteryl esters, including cholesteryl |
| | palmitate, oleate, linoleate, |
| | palmitoleate, linolenate, and myristate |
| Arthritis | Calcium pyrophosphate dihydrate |
| | Calcium hydrogen phosphate dihydrate |
| Cystinosis, Fanconi's syndrome, | Cystine |
| rickets, synovitis | |

VIII. Treatment of Cataracts

The lens of the vertebrate eye depends upon its shape, flexibility, transparency, and high refractive index (relative to the surrounding aqueous humor and vitreous humor) to perform its function: focusing light onto the retina. These properties in turn depend upon the properties of the highly specialized cells and densely packed proteins that constitute the lens. Proteins account for about 30-35%, and water about 65-70%, of the cellular mass of the lens; this compares to a water content of about 95% for most cells (Graw, J., (1997), “The crystallins: genes, proteins and diseases,” Biol Chem 378:1331-1346). The high protein density is responsible for the high refractive index of the lens. Such dense packing of the protein molecules requires specialized proteins and a high degree of organization to prevent interactions that may reduce transparency. In particular, the proteins must be kept in solution and be prevented from interacting with each other to form insoluble aggregates that could scatter light and reduce transparency.

The lens develops from epithelial cells that ultimately form its anterior surface. These cells migrate towards the posterior pole of the lens, and then greatly elongate to form closely aligned, nearly concentric rings of fiber cells. The nuclei and other organelles of the fiber cells degrade, increasing transparency. In cross-section, the fiber cells are hexagonal and are regularly ordered in a close-packed array. The cells are not, however, in direct contact with each other, but are separated by elaborate gap junctions supported by cylindrical structures called connexons, which are composed of connexin proteins. The gaps allow ions, second messengers, small metabolites, and other small molecules to reach the cells. Though the innermost part of the lens forms embryonically, fiber cells continue to be laid down on the periphery of the lens throughout life. The core of the adult lens appears to have little metabolic activity and little or no protein turnover, while the outer parts of the lens appear to have only a low level of metabolic activity and protein turnover. The constituents and structures of the human lens must thus be highly durable and stable to last the lifetime of an individual.

The cytoplasmic proteins of the lens fiber cells are predominately water soluble proteins called crystallins, particularly α-crystallins, β-crystallins, and γ-crystallins. The known structures and functions of the crystallins are described in many publications and will be considered only briefly here, primarily as they relate to cataract formation (for further information on crystallins see, e.g., Graw (supra); Francis, P. J. et al. (1999), “Lens biology, development and cataractogenesis, ” Trends in Genetics 15:191-196; Slingsby, C. et al. (1993), “Structure of the crystallins,” Eye 13:395-402; and Carver, J. A. (1999) “Probing the structure and interactions of crystalline proteins by NMR spectroscopy,” Progress in Retinal and Eye Research 18:431-462).

The α-crystallins are the most abundant cytoplasmic lens proteins. They are very large (800-1000 kDa) assemblages (oligomers) of about 20 copies each of αA- and aβ-crystallin subunits. The detailed structures of the α-crystallins have yet to be deduced. The α-crystallins are heat-shock proteins and function as molecular chaperones. In particular, within the lens they appear to bind denatured or otherwise damaged proteins, particularly β- and γ-crystallins, to keep them soluble and prevent them from aggregating; they may also prevent fructose-induced deactivation of lens enzymes (Graw, supra). The α-crystallins are also constituents, together with the cytoskeletal protein CP49, of the so-called “beaded filaments” observed within the lens by electron microscopy.

The β- and γ-crystallins are closely related to each other, but are not structurally or sequence-related to the α-crystallins. The β-crystallins form assemblages about 200 kDa in weight, consisting of about 10 subunit copies (with variants termed βA1-, βA2- , βA3, βA4- , βB1- , βB2- , and βB3-crystallin; β_{H} refers to a high molecular weight oligomeric aggregate and β_{L} refers to a low molecular weight oligomeric aggregate of these variants or portions of these variants; for more details see, e.g., Ajaz, M. S. et al. (1997), “Size of human lens β-crystallin aggregates are distinguished by N-terminal truncation of βB1”, J Biol Chem 272:11250-5). γ-crystallins (with variants designated as γA-, γB-, γC-, γD-, γE-, γF-, and γS-crystallin), in contrast, are monomeric, with molecular weights of about 21 kDa. Both β- and γ-crystallins appear to be structural and to contribute to raising the refractive index. The β- and particularly the γ-crystallins contain a large number of free SH groups, which could lead to disulfide bonding and resultant cross-inking and aggregation under oxidative stress. The high levels of reduced glutathione in the normal lens are thought to contribute to the inhibition of such reactions. γS-crystallin appears to have a role in preventing the cytoplasmic aggregation of other γ-crystallins (particularly the abundant γD-crystallin) that can lead to cataract formation (Liu, Q. et al. (1996) “Phase separation in aqueous solutions of lens γ-crystallins: special role of γs,” PNAS 93:377-382).

Cataracts, as previously discussed, are opacities in the lens. Although cataracts can take many forms and have many causes (e.g., genetic defects, oxidative stress, exposure to ultraviolet radiation (“welder's cataract”), exposure to microwave radiation, intraocular infection, diabetes, dehydration, mechanical injury), the great majority of cataracts are those that develop with age (senile cataracts). Studies of cataract formation in genetically altered mice, and in humans with injuries or with congenital genetic mutations, have revealed a number of mechanisms for cataract formation that may be applicable to senile cataract formation (see, e.g., Francis et al., supra and Graw, supra).

It has long been known that cataracts can be caused by, for example, ultraviolet radiation or oxidative stress. These factors can produce structural changes in the crystallins (Graw, supra). The α-crystallins appear to lose cysteine sulfhydryl groups and show an increase in the water-insoluble fraction under these stresses and with old age: these changes are linked to the formation of cataracts, likely due to a loss of the chaperone and protective effects of the degraded α-crystallins. Also, decreasing levels in the lens of reduced glutathione (and of other protective enzymes) with age allow disulfide bonding, cross-linking, and aggregation of β- and particularly γ-crystallins to occur under stress caused by, e.g., oxidation, dehydration, or ultraviolet radiation.

It has now been discovered that the aggregates of β- and γ-crystallins in senile and other cataracts are crystalline in nature. Kmoch et al. describe well-crystallized γD-crystallin crystals in a congenital cataract caused by a mutation of the γD-crystallin gene (Kmoch et al. (2000), “Link between a novel human γD-crystallin allele and a unique cataract phenotype explained by protein crystallography,” Human Molecular Genetics 9:1779-1786). Pande et al. expanded this observation to other congenital cataracts, and suggested that protein crystallization may be a general phenomenon in non-genetic cataracts (Pande et al. (2001) “Crystal cataracts: Human genetic cataract caused by protein crystallization,” PNAS 98:6116-6120). Petrographic microscopic observations using polarized light by the inventor have shown that human senile cataracts, unlike normal lenses, are polycrystalline. Since many of the crystallites are of a size on the order of the wavelength of visible light, they scatter the incident light, rendering the medium opaque.

Thus, a further preferred embodiment of the invention is the prevention or treatment of a cataract in the eye of a human patient by the administration to the patient of a therapeutically effective amount of an active agent effective to inhibit crystallization of at least one lenticular protein. The active agent may be administered in an ophthalmic formulation, to the patient's eye. If the active agent is orally active, it may be administered in a formulation suitable for oral drug administration. Other modes of administration are also possible, but are less preferred. The at least one lenticular protein is selected from the group consisting of α-crystallins, β-crystallins, γ-crystallins, and combinations thereof, and more typically will be selected from the group consisting of β_{H}-crystallins, γS-crystallins, γB-crystallins, and γD-crystallins, and combinations thereof.

It is to be understood that while the invention has been described in conjunction with the preferred specific embodiments thereof, the foregoing description as well as the examples that follow are intended to illustrate and not limit the scope of the invention. Other aspects, advantages, and modifications within the scope of the invention will be apparent to those skilled in the art to which the invention pertains.

All patents, patent applications, and publications mentioned herein are hereby incorporated by reference in their entireties.

The practice of the present invention will employ, unless otherwise indicated, conventional techniques of pharmaceutical formulation and the like, which are within the skill of the art. Such techniques are fully explained in the literature. In the following examples, efforts have been made to ensure accuracy with respect to numbers used (e.g., amounts, temperatures, etc.) but some experimental error and deviation should be accounted for. Unless otherwise indicated, temperature is in degrees Celsius and pressure is at or near atmospheric pressure at sea level. All reagents were obtained commercially unless otherwise indicated.

### EXAMPLE 1

In this example, cholesterol monohydrate, the crystalline component of atherosclerotic plaques, is evaluated for its ability to show affinity for other compounds. For this purpose, use is made of a combinatorial chemistry library, which is a combination of physical arrays of numerous small vessels, each containing a solution of a different chemical compound. In this example, the compounds making up the arrays are limited to those of heterocyclic structure.

A volume of 0.1 mL of a 10 mg/mL dimethylsulfoxide solution of each compound is added by micropipette to a vessel in an array. Each array contains 80 vessels. Following this, 0.1 mL of a 10 mg/mL dimethylsulfoxide solution of cholesterol monohydrate is added by micropipette to each vessel in ten (10) arrays, and possible affinities between the ingredients of each vessel are evaluated by two procedures: changes in refractive index of the solutions, and shifts in the spectrophotometric absorption spectrum in the visible range are detected following addition of 0.1 mL of a 0.02 mg/mL solution of rosaniline. Changes in either of these parameters signifies an affinity. Of some seven affinities thus identified, two are found to also reduce the severity of atheroma formation in cholesterol-fed rabbits. Separately, these two compounds are administered orally to six New Zealand white rabbits at a dose of 250 mg per animal per day for three weeks, concomitant with the feeding of these animals, plus 6 additional rabbits, an atherogenic diet. Examination of the aortas of all animals following autopsy during the fourth week shows a significant decrease in the degree of intimal atheroma formation only in the 6 animals that had received either of the two compounds. Following this demonstration of efficacy, the two compounds are then first shown to be free of toxicity, and then shown by radiological examination to be effective in decreasing the rate of formation of atherosclerotic plaques in high blood-cholesterol human patients, when administered orally in 200 mg capsules, twice daily, for a period of eight weeks.

### EXAMPLE 2

The procedure of Example 1 is repeated, except that the two compounds identified as efficacious in reducing the rate of atheroma formation are shown to reduce the severity of biliary calculi (gallstones) in afflicted patients, when administered orally twice daily, in 500 mg capsules.

### EXAMPLE 3

In this example, a quantity of human senile cataracts, following surgical removal, are collected, lyophilized, reduced to a powder using a blender/homogenizer, and the powder equilibrated with dimethylsulfoxide at a temperature of 37° C. for two hours. Supernatant solution (0.1 mL) is added to each of the vessels of a 300-plate (80 compounds per plate) combinatorial chemistry library, and the library evaluated for the occurrence of affinities (hybridizations). Some seven compounds of the library are found to demonstrate affinities as measured by a change in refractive index of the vessel's contents, using an Abbe refractometer. Each of these compounds is then found by slit-lamp examination to be effective in halting the progression of posterior subcapsular cataracts in human cataract patients when administered twice daily in the form of 5% sterile aqueous eyedrops.

### EXAMPLE 4

Reference is made to two chemical compounds: pure cholesterol (C-pure), and modified cholesterol (C-mod). C-mod is made by partial thermal degradation of C-pure. As a consequence, C-mod is structurally very similar, but not identical, to C-pure. In the first of three steps to this example, a quantity of C-mod is produced by heating 10 grams of dry C-pure in the presence of oxygen for 30 minutes at 134° C., which is a temperature that is 15° below the melting point of the pure substance. In a second step to this example, 10 mg of C-pure is added to one liter of distilled water. The resulting suspension is then stirred at 50° C. for two hours, followed by stirring at 37° C. for 6 hours. The concentration of solute in solution at 37° is then determined by an HPLC analytical method. This value is the water-solubility of pure cholesterol at 37° C., and is found to be 0.45 mg/100 mL. In the third and final step to this example, the process of the second step is repeated with 10 mg of C-pure in one liter of distilled water, and 10 mg of C-mod is also added to the initial suspension. In this case, HPLC determination of the concentration of C-pure in the supernatant solution results in a finding of 1.2 mg/100 mL. It is previously demonstrated that the HPLC method used is specific to C-pure, and is not influenced by the presence of C-mod. This increase in apparent solubility of C-pure is considered to be a consequence of the selective inhibition of crystal nucleation and growth, due to the presence of molecules that are similar to but not identical to those of the solute. It is subsequently found that 250 mg of C-mod taken orally per day by atherosclerotic patients slows or halts the progress of developing atheromas, as determined by radiologic examination.

### EXAMPLE 5

This example is identical to Example 1, except that uric acid, the crystalline material in the joints and tendons of hyperuricemia (gout) patients, is substituted for cholesterol monohydrate, and affinities are sought in a combinatorial chemistry library using refractive index changes as an indication of affinity. Of some 12 affinities identified, three are found to reduce the symptoms of pain and swelling in hyperuricemic patients, following oral administration twice daily of any of the three compounds in 250 mg capsules for from two to four months. Alternatively, two of the three compounds are found to be ionizable, and daily administration of either of these agents through iontophoretic application directly to the affected joints of the patients is seen to be effective in two to four weeks.

### EXAMPLE 6

This example is identical to Example 5, except that sodium oxalate is substituted for uric acid in the affinity search. Of some nine compounds displaying affinity, two are then found to be effective in reducing symptomatic sensation of pain experienced by patients with renal calculi (kidney stones), following four months of treatment involving twice-daily oral administration of 300 mg capsules.

I claim:

1. A method for simultaneously screening a plurality of candidate compounds for their ability to inhibit formation of a crystalline structure of a selected biomolecule that is endogenous to the human body, wherein in vivo formation of the crystalline structure is an adverse event resulting in at least one medical pathology selected from diseases, disorders, and other undesirable physiological conditions, the method comprising:
(a) providing a combinatorial library of a plurality of different candidate compounds each attached to a different site on a substrate;
(b) contacting the library of candidate compounds with the selected biomolecule under conditions effective to facilitate formation of said crystalline structure in the absence of any inhibitors;
(c) identifying candidate compounds for which the biomolecule has affinity by determining which candidate compounds have become physically associated with the biomolecule during step (b); and
(d) selecting the candidate compounds identified in step (c) as potential inhibitors of in vivo formation of the crystalline structure of the biomolecule.

2. The method of claim 1, wherein the different candidate compounds are selected so that they are structurally similar but nonidentical to the biomolecule.

3. The method of claim 2, wherein formation of the crystalline structure comprises formation of fibrils.

4. The method of claim 2, wherein formation of the crystalline structure comprises formation of ocular cataracts.

5. The method of claim 1, wherein the biomolecule is selected from the group consisting of peptidic molecules, sterols, uric acid, uric acid salts, and calcium salts.

6. The method of claim 5, wherein the biomolecule is a peptidic molecule.

7. The method of claim 6, wherein the candidate compounds are independently selected from the group consisting of:
(a) oligopeptide fragments contained within the peptidic molecule; and
(b) analogs of the peptidic molecule wherein the peptidic molecule is modified by (i) substitution of one or more amino acids, (ii) deletion of one or more amino acids, (iii) insertion of one or more amino acids, (iv) an N-terminal modification, (v) a C-terminal modification, or combinations thereof.

8. The method of claim 3, wherein the biomolecule is a peptidic molecule.

9. The method of claim 8, wherein the candidate compounds are independently selected from the group consisting of:
(a) oligopeptide fragments contained within the peptidic molecule; and
(b) analogs of the peptidic molecule wherein the peptidic molecule is modified by (i) substitution of one or more amino acids, (ii) deletion of one or more amino acids, (iii) insertion of one or more amino acids, (iv) an N-terminal modification, (v) a C-terminal modification, or combinations thereof.

10. The method of claim 4, wherein the biomolecule is selected from the group consisting of peptidic molecules, sterols, uric acid, uric acid salts, and calcium salts.

11. The method of claim 10, wherein the biomolecule is a peptidic molecule.

12. The method of claim 11, wherein the candidate compounds are independently selected from the group consisting of:
(a) oligopeptide fragments contained within the peptidic molecule; and
(b) analogs of the peptidic molecule wherein the peptidic molecule or oligopeptide fragments thereof are modified by (i) substitution of one or more amino acids, (ii) deletion of one or more amino acids, (iii) insertion of one or more amino acids, (iv) an N-terminal modification, (v) a C-terminal modification, or combinations thereof.

13. The method of claim 8, wherein the biomolecule is selected from the group consisting of amyloid and amyloid-β.

14. The method of claim 13, wherein the biomolecule is amyloid-β.

15. The method of claim 14, wherein the amyloid-β is selected from the group consisting of amyloid-β (1-40), amyloid-β (1-42), and amyloid-β (1-43).

16. The method of claim 14, wherein the candidate compounds are independently selected from the group consisting of:
(a) oligopeptide fragments contained within amyloid-β; and
(b) amyloid-β analogs wherein amyloid-β or oligopeptide fragments thereof are modified by (i) substitution of one or more amino acids, (ii) deletion of one or more amino acids, (iii) insertion of one or more amino acids, (iv) an N-terminal modification, (v) a C-terminal modification, or combinations thereof.

17. The method of claim 16, wherein the candidate compounds are oligopeptide fragments contained within amyloid-β.

18. The method of claim 8, wherein the biomolecule is prion protein (PrP) or an oligopeptide fragment thereof.

19. The method of claim 18, wherein candidate compounds are independently selected from the group consisting of PrP analogs wherein PrP or oligopeptide fragments thereof are modified by (i) substitution of one or more amino acids, (ii) deletion of one or more amino acids, (iii) insertion of one or more amino acids, (iv) an N-terminal modification, (v) a C-terminal modification, or combinations thereof.

20. The method of claim 18, wherein the biomolecule is PrP.

21. The method of claim 18, wherein the biomolecule is a PrP fragment.

22. The method of claim 21, wherein the PrP fragment is PrP96-111.

23. The method of claim 8, wherein the biomolecule is fibrillar collagen.

24. The method of claim 8, wherein the biomolecule is fibrillin.

25. The method of claim 6, wherein the biomolecule is a lenticular protein.

26. The method of claim 25, wherein the biomolecule is selected from beta, gamma, alpha crystallins, and combinations thereof.

27. The method of claim 26, wherein the biomolecule is selected from beta and gamma crystallins.

28. The method of claim 26, wherein the biomolecule is comprised of a mixture of beta and gamma crystallins.

29. The method of claim 27, wherein the biomolecule is selected from the group consisting of β_{H}-crystallins, γS-crystallins, γC-crystallins, γD-crystallins, and combinations thereof.

30. The method of claim 6, wherein the biomolecule is cystic fibrosis transmembrane conductance regulator (“CFTR”) protein.

31. The method of claim 6, wherein the biomolecule is a Charcot-Leyden protein.

32. The method of claim 6, wherein the biomolecule is selected from the group consisting of cystine, hemoglobin, hematoidin, cryoglobulins, and immunoglobulins.

33. The method of claim 5, wherein the biomolecule is a sterol.

34. The method of claim 33, wherein the biomolecule is cholesterol, cholesterol monohydrate, or a cholesteryl ester.

35. The method of claim 5, wherein the biomolecule is a calcium salt.

36. The method of claim 35, wherein the calcium salt is calcium oxalate, calcium oxalate monohydrate, hydroxyapatite, octacalcium phosphate, tricalcium phosphate, calcium hydrogen phosphate dihydrate, calcium pyrophosphate, or calcium pyrophosphate dihydrate.

37. The method of claim 10, wherein the biomolecule is uric acid or a salt thereof.

38. The method of claim 37, wherein the biomolecule is monosodium urate or monosodium urate monohydrate.

39. The method of claim 1, wherein the biomolecule is labeled with a detectable label, and step (c) is carried out by detecting the presence of the label at any of the different sites on the substrate.

40. The method of claim 39, wherein the label is a fluorescent label, and is detected using fluorescence spectroscopy.

41. The method of claim 39, wherein the label is detectable using ultraviolet spectroscopy.

42. The method of claim 39, wherein the label is a colored species, and is visually detectable.

43. The method of claim 39, wherein the label is detectable by virtue of an altered refractive index relative to the unlabeled biomolecule.

44. The method of claim 1, wherein steps (a) through (c) are successively repeated with a plurality of different biomolecules.

45. A method for identifying a compound capable of inhibiting formation of a crystalline structure of a selected biomolecule that is endogenous to the human body, wherein in vivo formation of the crystalline structure is an adverse event resulting in at least one medical pathology selected from diseases, disorders, and other undesirable physiological conditions, the method comprising:
(a) providing a combinatorial library of a plurality of different candidate compounds each attached to a different site on a substrate;
(b) contacting the library of candidate compounds with the selected biomolecule under conditions allowing the biomolecule to contact each of the candidate compounds;
(c) identifying candidate compounds for which the biomolecule has affinity by determining which candidate compounds have become physically associated with the biomolecule during step (b);
(d) selecting the candidate compounds identified in step (c) as potential inhibitors of in vivo formation of the crystalline structure of the biomolecule;
(e) for at least one candidate compound selected in step (d), contacting the biomolecule with the selected candidate compound under conditions effective to facilitate formation of said crystalline structure in the absence of the selected candidate compound; and
(f) determining whether the crystalline structure is formed in step (e), and if the crystalline structure has not been formed, identifying the selected candidate compound as a compound capable of inhibiting formation of said crystalline structure.

46. The method of claim 45, wherein steps (e) and (f) are successively conducted with each of the candidate compounds selected in (d).

47. The method of claim 45, wherein the plurality of different candidate compounds is selected so that each compound is structurally similar but nonidentical to the biomolecule.

48. The method of claim 47, wherein formation of the crystalline structure comprises formation of fibrils.

49. The method of claim 47, wherein formation of the crystalline structure comprises formation of ocular cataracts.

50. The method of claim 45, wherein the biomolecule is selected from the group consisting of peptidic molecules, sterols, uric acid, uric acid salts, and calcium salts.

51. The method of claim 50, wherein the biomolecule is a peptidic molecule.

52. The method of claim 51, wherein the candidate compounds are independently selected from the group consisting of:
(a) oligopeptide fragments contained within the peptidic molecule; and
(b) analogs of the peptidic molecule wherein the peptidic molecule is modified by (i) substitution of one or more amino acids, (ii) deletion of one or more amino acids, (iii) insertion of one or more amino acids, (iv) an N-terminal modification, (v) a C-terminal modification, or combinations thereof.

53. The method of claim 48, wherein the biomolecule is a peptidic molecule.

54. The method of claim 53, wherein the candidate compounds are independently selected from the group consisting of:
(a) oligopeptide fragments contained within the peptidic molecule; and
(b) analogs of the peptidic molecule wherein the peptidic molecule is modified by (i) substitution of one or more amino acids, (ii) deletion of one or more amino acids, (iii) insertion of one or more amino acids, (iv) an N-terminal modification, (v) a C-terminal modification, or combinations thereof.

55. The method of claim 49, wherein the biomolecule is selected from the group consisting of peptidic molecules, sterols, uric acid, uric acid salts, and calcium salts.

56. The method of claim 55, wherein the biomolecule is a peptidic molecule.

57. The method of claim 56, wherein the candidate compounds are independently selected from the group consisting of:
(a) oligopeptide fragments contained within the peptidic molecule; and
(b) analogs of the peptidic molecule wherein the peptidic molecule or oligopeptide fragments thereof are modified by (i) substitution of one or more amino acids, (ii) deletion of one or more amino acids, (iii) insertion of one or more amino acids, (iv) an N-terminal modification, (v) a C-terminal modification, or combinations thereof.

58. The method of claim 53, wherein the biomolecule is selected from the group consisting of amyloid and amyloid-β.

59. The method of claim 58, wherein the biomolecule is amyloid-β.

60. The method of claim 59, wherein the amyloid-β is selected from the group consisting of amyloid-β (1-40), amyloid-β (1-42), and amyloid-β (1-43).

61. The method of claim 60, wherein the candidate compounds are independently selected from the group consisting of:
(a) oligopeptide fragments contained within amyloid-β; and
(b) amyloid-β analogs wherein amyloid-β or oligopeptide fragments thereof are modified by (i) substitution of one or more amino acids, (ii) deletion of one or more amino acids, (iii) insertion of one or more amino acids, (iv) an N-terminal modification, (v) a C-terminal modification, or combinations thereof.

62. The method of claim 61, wherein the candidate compounds are oligopeptide fragments contained within amyloid-β.

63. The method of claim 53, wherein the biomolecule is prion protein (PrP) or an oligopeptide fragment thereof.

64. The method of claim 63, wherein candidate compounds are independently selected from the group consisting of PrP analogs wherein PrP or oligopeptide fragments thereof are modified by (i) substitution of one or more amino acids, (ii) deletion of one or more amino acids, (iii) insertion of one or more amino acids, (iv) an N-terminal modification, (v) a C-terminal modification, or combinations thereof.

65. The method of claim 63, wherein the biomolecule is PrP.

66. The method of claim 63, wherein the biomolecule is a PrP fragment.

67. The method of claim 66, wherein the PrP fragment is PrP96-111.

68. The method of claim 53, wherein the biomolecule is fibrillar collagen.

69. The method of claim 53, wherein the biomolecule is fibrillin.

70. The method of claim 51, wherein the biomolecule is a lenticular protein.

71. The method of claim 70, wherein the biomolecule is selected from beta, gamma, alpha crystallins, and combinations thereof.

72. The method of claim 71, wherein the biomolecule is selected from beta and gamma crystallins.

73. The method of claim 71, wherein the biomolecule is comprised of a mixture of beta and gamma crystallins.

74. The method of claim 72, wherein the biomolecule is selected from the group consisting of β_{H}-crystallins, γS-crystallins, γC-crystallins, γD-crystallins, and combinations thereof.

75. The method of claim 51, wherein the biomolecule is cystic fibrosis transmembrane conductance regulator (“CFTR”) protein.

76. The method of claim 51, wherein the biomolecule is a Charcot-Leyden protein.

77. The method of claim 51, wherein the biomolecule is selected from the group consisting of cystine, hemoglobin, hematoidin, cryoglobulins, and immunoglobulins.

78. The method of claim 50, wherein the biomolecule is a sterol.

79. The method of claim 78, wherein the biomolecule is cholesterol, cholesterol monohydrate, or a cholesteryl ester.

80. The method of claim 55 wherein the biomolecule is a calcium salt.

81. The method of claim 80, wherein the calcium salt is calcium oxalate, calcium oxalate monohydrate, hydroxyapatite, octacalcium phosphate, tricalcium phosphate, calcium hydrogen phosphate dihydrate, calcium pyrophosphate, or calcium pyrophosphate dihydrate.

82. The method of claim 55, wherein the biomolecule is uric acid or a salt thereof.

83. The method of claim 82, wherein the biomolecule is monosodium urate or monosodium urate monohydrate.

84. The method of claim 45, wherein the biomolecule is labeled with a detectable label, and step (c) is carried out by detecting the presence of the label at any of the different sites on the substrate.

85. The method of claim 84, wherein the label is a fluorescent label, and is detected using fluorescence spectroscopy.

86. The method of claim 84, wherein the label is detectable using ultraviolet spectroscopy.

87. The method of claim 84, wherein the label is a colored species, and is visually detectable.

88. The method of claim 84, wherein the label is detectable by virtue of an altered refractive index relative to the unlabeled biomolecule.

89. The method of claim 45, wherein steps (a) through (c) are successively repeated with a plurality of different biomolecules.

90. A therapeutic agent comprising the selected candidate compound identified in step (f) of claim 45.

91. A method for treating a patient afflicted with a medical pathology associated with in vivo formation of a crystalline structure of a biomolecule that is endogenous to the human body, the method comprising:
administering to the patient a therapeutically effective amount of the compound of claim 47.

92. The method of claim 91, wherein formation of the crystalline structure comprises fibril formation.

93. The method of claim 91, wherein the medical pathology is cataract formation, and the biomolecule is a lenticular protein.

94. A method for preventing or treating cataract formation in the eye of a human patient, comprising administering to the patient a therapeutically effective amount of an active agent effective to inhibit crystallization of at least one lenticular protein.

95. The method of claim 94, wherein the active agent is administered in an ophthalmic formulation to the patient's eye.

96. The method of claim 94, wherein the active agent is orally active, and is orally administered in a formulation suitable for oral drug administration.

97. The method of claim 94, wherein the at least one lenticular protein is selected from the group consisting of beta crystallins, gamma crystallins, alpha crystallins, and combinations thereof.

98. The method of claim 97, wherein the at least one lenticular protein is selected from the group consisting of βH-crystallins, γS-crystallins, γC-crystallins, and γD-crystallins, and combinations thereof.

99. A method for simultaneously screening a plurality of candidate compounds for their ability to inhibit formation of a pathogenic mass composed of a crystalline structure of at least one molecular component and associated with a particular medical pathology, the method comprising:
(a) preparing a particulate suspension of the at least one molecular component of the pathogenic mass, by obtaining a pathogenic mass that has been freshly removed from a human patient, lyophilizing and triturating the freshly removed mass, and admixing the lyophilized, triturated mass with an inert organic solvent;
(b) contacting a combinatorial library of candidate compounds with the particulate suspension under conditions effective to facilitate formation of said crystalline structure in the absence of any crystalline structure formation inhibitors; and
(c) identifying candidate compounds for which one or more molecular components of the pathogenic mass have affinity by determining which candidate compounds have become physically associated with a component of the particulate suspension during step (b).

100. The method of claim 99, wherein the pathogenic mass is a cataract.

101. The method of claim 99, wherein the pathogenic mass is atherosclerotic plaque.

102. The method of claim 99, wherein the pathogenic mass is neuritic plaque.

103. The method of claim 99, wherein the pathogenic mass is dendritic plaque.

104. The method of claim 99, wherein the pathogenic mass is a gallstone.

105. The method of claim 99, wherein the pathogenic mass is a kidney stone.

106. A method for identifying a compound capable of inhibiting formation of a pathogenic mass composed of a crystalline structure of at least one molecular component and associated with a particular medical pathology, the method comprising:
(a) carrying out the method of claim 99;
(b) for at least one candidate compound identified in step (c), contacting the particulate suspension with the selected candidate compound under conditions effective to facilitate formation of said crystalline structure in the absence of any crystalline structure formation inhibitors; and
(c) determining whether the crystalline structure is formed in step (e), and if the crystalline structure has been formed, identifying the selected candidate compound as a compound capable of inhibiting formation of the pathogenic mass.

107. The method of claim 106, wherein the pathogenic mass is a cataract.

108. The method of claim 106, wherein the pathogenic mass is atherosclerotic plaque.

109. The method of claim 106, wherein the pathogenic mass is neuritic plaque.

110. The method of claim 106, wherein the pathogenic mass is dendritic plaque.

111. The method of claim 106, wherein the pathogenic mass is a gallstone.

112. The method of claim 106, wherein the pathogenic mass is a kidney stone.

113. A method for identifying a compound capable of inhibiting formation of a crystalline structure of a selected biomolecule that is endogenous to the human body, wherein in vivo formation of the crystalline structure is an adverse event resulting in at least one medical pathology selected from diseases, disorders, and other undesirable physiological conditions, the method comprising:
(a) preparing a potential inhibitor by chemically modifying the biomolecule with heat and/or a chemical reactant so as to provide an analog of the biomolecule having at least one difference in molecular structure relative to the unmodified biomolecule;
(b) contacting the biomolecule with the analog under conditions effective to facilitate formation of said crystalline structure in the absence of any crystalline structure formation inhibitors; and
(c) determining whether the crystalline structure is formed in step (b), and if the crystalline structure has not been formed, identifying the analog as a compound capable of inhibiting formation of said crystalline structure.

114. The method of claim 113, wherein steps (a) through (c) are repeated in succession.
